(19) United States
(12) Patent Application Publication
(10) Pub. No.: US 2005/0065563 A1
(43) Pub. Date: Mar. 24, 2005

(54) Paced ventilation therapy by an implantable cardiac device

(76) Inventor: Avram Scheiner, Vadnais Heights, MN (US)

Correspondence Address: Schwegman, Lundberg, Woessner & Kluth, P.A., P.O. Box 2938, Minneapolis, MN, 55402, US

(21) Appl. No.: 10/669,154

(22) Filed: Sep. 23, 2003

(51) Int. Cl. A61N 1/362

(52) U.S. Cl. 607/9

(57) ABSTRACT

An implantable cardiac rhythm management device for treating tachyarrhythmias such as ventricular fibrillation which also has the capability of detecting and treating respiratory arrest. The device restores respiratory function by electrically stimulating the diaphragm with pacing electrodes which may be normally used by the device for cardiac pacing. In response to detection of ventricular fibrillation and respiratory arrest, the device delivers shock pulses to terminate the fibrillation and diaphragmatic pacing pulses to restore breathing.

### FIELD OF THE INVENTION

This invention pertains to cardiac rhythm management devices such as pacemakers and implantable cardioverter/defibrillators.

### BACKGROUND

Tachyarrhythmias are abnormal heart rhythms characterized by a rapid heart rate. Examples of ventricular tachyarrhythmias include ventricular tachycardia (VT) and ventricular fibrillation (VF). Both ventricular tachycardia and ventricular fibrillation can be hemodynamically compromising, and both can be life-threatening. Ventricular fibrillation, however, causes circulatory arrest within seconds and is the most common cause of sudden cardiac death. Cardioversion (an electrical shock delivered to the heart synchronously with an intrinsic depolarization) and defibrillation (an electrical shock delivered without such synchronization) can be used to terminate most tachyarrhythmias, including VT and VF. Both defibrillation and cardioversion terminate a tachyarrhythmia by depolarizing a critical mass of myocardial cells so that the remaining myocardial cells are not sufficient to sustain the tachyarrhythmia. Implantable cardioverter/defibrillators (ICDs) provide electrotherapy by delivering a shock pulse to the heart when fibrillation is detected by the device. An ICD is an electronic device containing circuitry for sensing cardiac activity and for generating a shock pulse when a tachyarrhythmia is detected. The device is usually implanted into the chest or abdominal wall and connected to electrodes used for shocking and sensing by transvenously passed leads.

When cardiac arrest occurs due to ventricular fibrillation, both the heart and the brain are deprived of oxygen as a result of circulatory insufficiency. If an ICD is successful in terminating the ventricular fibrillation promptly, both cardiac and brain function are restored as circulation returns. If the circulatory arrest is not promptly terminated, however, respiratory arrest can occur secondarily due to the neural centers controlling respiration being affected by ischemia. Respiratory arrest can complicate treatment of the ventricular fibrillation because insufficiently oxygenated blood can make ventricular fibrillation more difficult to terminate and may prevent reversal of the respiratory arrest even if circulation to the brain is returned. Manual techniques for resuscitating individuals suffering from cardiac arrest thus include both chest compression for restoring circulation and forcing air into the lungs. It would be advantageous for an ICD to also have a capability for treating respiratory arrest.

### SUMMARY

The present invention relates to an implantable cardiac rhythm management device for treating tachyarrhythmias such as ventricular fibrillation which also has the capability of detecting and treating respiratory arrest. The device restores respiratory function by electrically stimulating the diaphragm with pacing electrodes which may be normally used by the device for cardiac pacing. In response to detection of ventricular fibrillation and respiratory arrest, the device delivers shock pulses to terminate the fibrillation and diaphragmatic pacing pulses to restore breathing. The device may also deliver diaphragmatic pacing pulses after cardiac function is restored if the respiratory arrest persists.

### BRIEF DESCRIPTION OF THE DRAWINGS

FIG. 1 is a block diagram of an exemplary implantable cardiac device.

FIG. 2 illustrates an exemplary algorithm for treating ventricular fibrillation accompanied by respiratory arrest.

### DETAILED DESCRIPTION

As described above, hypoxia of the neural centers controlling breathing due to cardiac arrest may complicate resuscitation with shock therapy alone. In accordance with the present invention, an implantable cardiac rhythm management device may be configured with the capability of both delivering cardiac shock therapy to treat ventricular fibrillation and stimulating the diaphragm to force air into and out of the lungs if no spontaneous breathing is detected. The following are descriptions of an exemplary hardware platform and algorithm for implementing the technique.

1. Exemplary Implantable Device Description

Cardiac rhythm management devices are implantable battery-powered devices that provide electrical stimulation to selected chambers of the heart in order to treat disorders of cardiac rhythm. Such devices are usually implanted subcutaneously on the patient's chest and connected to electrodes by leads threaded through the vessels of the upper venous system into the heart. An electrode can be incorporated into a sensing channel that generates an electrogram signal representing cardiac electrical activity at the electrode site and/or incorporated into a pacing or shocking channel for delivering pacing or shock pulses to the site.

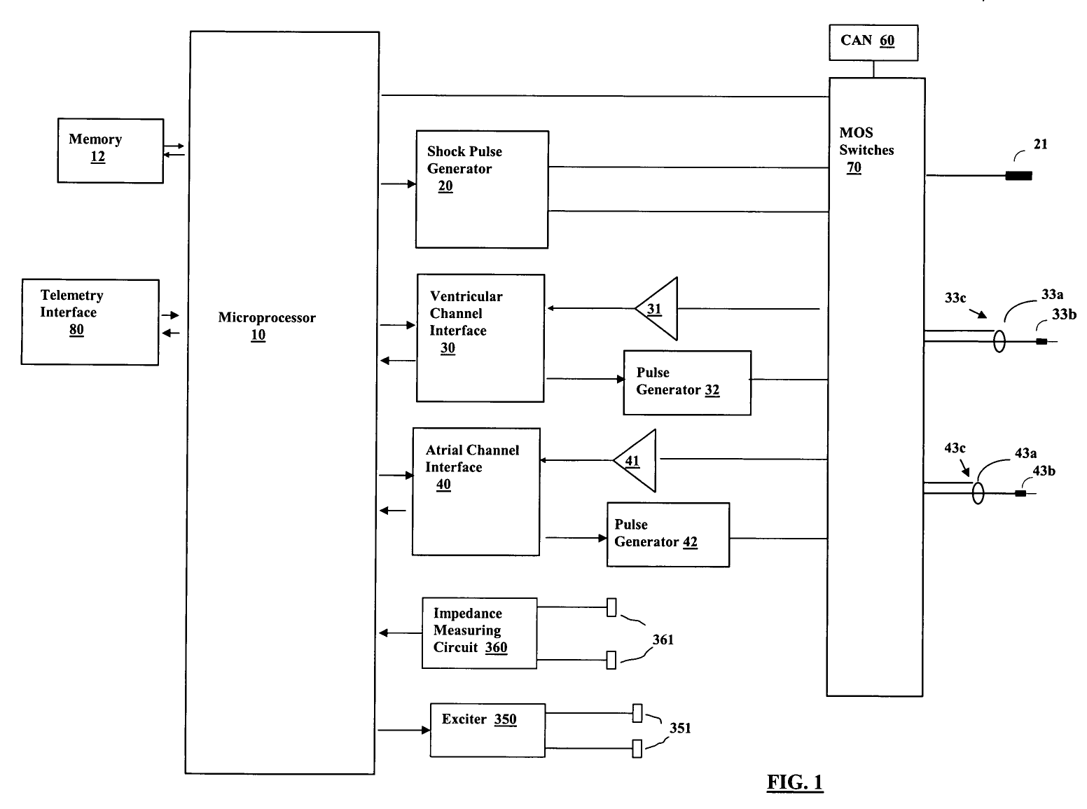

A block diagram of an implantable cardiac rhythm management device is shown in FIG. 1. The controller of the device is made up of a microprocessor 10 communicating with a memory 12 via a bidirectional data bus, where the memory 12 typically comprises a ROM (read-only memory) for program storage and a RAM (random-access memory) for data storage. The controller could be implemented by other types of logic circuitry (e.g., discrete components or programmable logic arrays) using a state machine type of design, but a microprocessor-based system is preferable. As used herein, the programming of a controller should be taken to refer to either discrete logic circuitry configured to perform particular functions or to executable code stored in memory or other storage medium. The controller is capable of operating the device so as to deliver a number of different therapies in response to detected cardiac activity. A telemetry interface 80 is also provided for enabling the controller to communicate with an external programmer.

The embodiment shown in FIG. 1 has two sensing/pacing channels, where a pacing channel is made up of a pulse generator connected to an electrode while a sensing channel is made up of the sense amplifier connected to an electrode. A MOS switch matrix 70 controlled by the microprocessor is used to switch the electrodes from the input of a sense amplifier to the output of a pulse generator. The switch matrix 70 also allows the sensing and pacing channels to be configured by the controller with different combinations of the available electrodes. The channels may be configured as either atrial or ventricular channels. In an example configuration, an atrial sensing/pacing channel includes ring electrode 43a and tip electrode 43b of bipolar lead 43c, sense amplifier 41, pulse generator 42, and a channel interface 40. A ventricular sensing/pacing channel includes ring electrode 33a and tip electrode 33b of bipolar lead 33c, sense amplifier 31, pulse generator 32, and a channel interface 30. The channel interfaces communicate bi-directionally with a port of microprocessor 10 and may include analog-to-digital converters for digitizing sensing signal inputs from the sensing amplifiers, registers that can be written to for adjusting the gain and threshold values of the sensing amplifiers, and registers for controlling the output of pacing pulses and/or changing the pacing pulse energy (i.e., the pulse amplitude and/or duration). In the illustrated embodiment, the device is equipped with bipolar leads that include two electrodes which are used for outputting a pacing pulse and/or sensing intrinsic activity. Other embodiments may employ unipolar leads with single electrodes for sensing and pacing. The switch matrix 70 may configure a channel for unipolar sensing or pacing by referencing an electrode of a unipolar or bipolar lead with the device housing or can 60.

A shock pulse generator 20 is also interfaced to the controller for delivering defibrillation shocks through electrodes selected by the switch matrix. For example, a shock pulse may be delivered between a shocking coil electrode 21 and the can 60. ICDs for delivering ventricular defibrillation shocks typically use an output capacitor that is charged from the battery with an inductive boost converter to deliver the shock pulse. When ventricular fibrillation is detected, the ICD charges up the output capacitor to a predetermined value for delivering a shock pulse of sufficient magnitude to convert the fibrillation (i.e., the defibrillation threshold). The output capacitor is then connected to the shock electrodes disposed in the heart to deliver the shock pulse. Since ventricular fibrillation is immediately life threatening, these steps are performed in rapid sequence with the shock pulse delivered as soon as possible.

The controller 10 controls the overall operation of the device in accordance with programmed instructions stored in memory. The controller 10 interprets electrogram signals from the sensing channels in order to control the delivery of paces in accordance with a pacing mode and/or deliver shock therapy in response to detection of a tachyarrhythmia such as ventricular fibrillation. The sensing circuitry of the device generates atrial and ventricular electrogram signals from the voltages sensed by the electrodes of a particular channel. An electrogram is analogous to a surface ECG and indicates the time course and amplitude of cardiac depolarization that occurs during either an intrinsic or paced beat. When an electrogram signal in an atrial or sensing channel exceeds a specified threshold, the controller detects an atrial or ventricular sense, respectively, which may also be referred to as a P-wave or R-wave in accordance with its representation in a surface ECG. The controller may use sense signals in pacing algorithms in order to trigger or inhibit pacing and to derive heart rates and by measuring the time intervals between senses.

Also interfaced to the controller is a thoracic impedance channel with includes an exciter 350 and an impedance measuring circuit 360. The exciter supplies excitation current of a specified amplitude (e.g., as a pulse waveform with constant amplitude) to excitation electrodes 351 that are disposed in the thorax. Voltage sense electrodes are disposed in a selected region of the thorax so that the potential difference between the electrodes while excitation current is supplied is representative of the transthoracic impedance between the voltage sense electrodes. In other embodiments, electrodes normally used for sensing and/or pacing can be switched by the switch matrix and used as voltage sense and/or excitation electrodes. The conductive housing or can may also be used as one of the voltage sense electrodes. The impedance measuring circuitry 360 processes the voltage sense signal from the voltage sense electrodes 361 to derive the impedance signal. Further processing of the impedance signal allows the derivation of signal representing respiratory activity and/or cardiac blood volume, depending upon the location the voltage sense electrodes in the thorax. (See, e.g., U.S. Pat. Nos. 5,190,035 and 6,161,042, assigned to the assignee of the present invention and hereby incorporated by reference.) For purposes of the present invention, the voltage sense electrodes are disposed so as to detect respiratory activity. The resulting voltage sense signal can then be used to derive minute ventilation for rate-adaptive pacing or, as explained below, to detect respiratory arrest.

2. Diaphragmatic Pacing for Treating Respiratory Arrest

As described above, internal electrodes for delivering cardiac pacing pulses are disposed near the heart by means of transvenously passed leads which connect the electrodes to the pulse generator(s) of the implanted cardiac device. Such electrodes may be disposed, for example, in the right atrium, the right ventricle, the coronary sinus, or a cardiac vein. The left phrenic nerve, which provides innervation for the diaphragm, arises from the cervical spine and descends to the diaphragm through the mediastinum where the heart is situated. As it passes the heart, the left phrenic nerve courses along the pericardium, superficial to the left atrium and left ventricle. Because of its proximity to the electrodes used for pacing, the nerve can be stimulated by a pacing pulse. The result is contraction of the diaphragm so that air is forced into the lungs.

In order to cause a diaphragmatic contraction, the energy of a pacing pulse must be greater than that required to cause an atrial or ventricular contraction, typically on the order of 10 to 30 volts. If ventricular fibrillation is present, the heart will be unaffected by such a pacing pulse. If ventricular fibrillation and respiratory arrest are both detected, the device may therefore deliver diaphragmatic pacing pulses during the time that the output capacitor for delivering a shock pulse is being charged. If, after the ventricular fibrillation is successfully terminated by the shock therapy, respiratory arrest is still present, diaphragmatic pacing pulses should be delivered in a manner which does not interfere with the ventricular rhythm and does not present a risk of re-triggering ventricular fibrillation. When respiratory arrest is present while a non-fibrillating ventricular rhythm is present, a diaphragmatic pacing pulse is delivered during the ventricular refractory period after a ventricular sense. Preferably, the diaphragmatic pacing pulses are delivered during the absolute refractory period which occurs shortly after each ventricular sense.

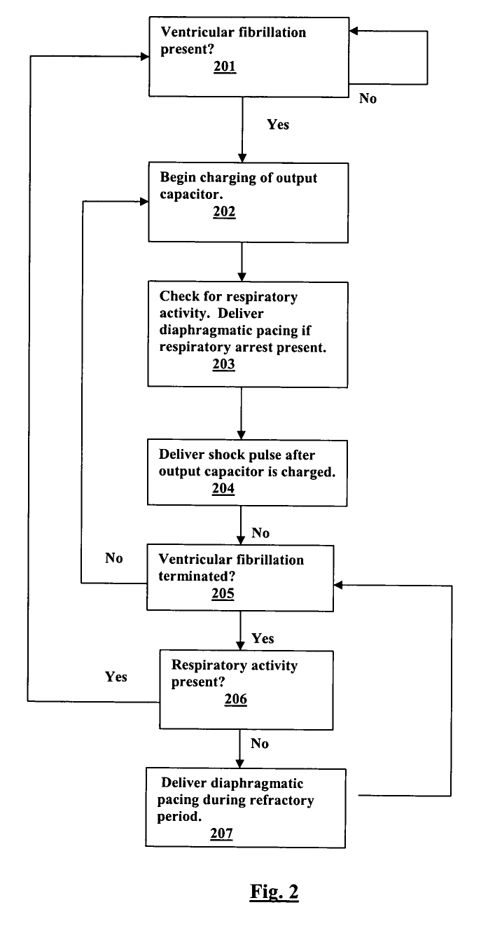

FIG. 2 illustrates an exemplary algorithm by which an implantable device configured for diaphragmatic pacing may treat ventricular fibrillation accompanied by respiratory arrest. At step 201, the device monitors a ventricular sensing channel in order to detect if ventricular fibrillation is present. If ventricular fibrillation is present, the device proceeds to step 202 to begin the process of charging the output capacitor for delivering a shock pulse. At the same time, the device also checks the thoracic impedance channel for the presence of respiratory activity at step 203. If respiratory arrest is also present, the device delivers one or more diaphragmatic pacing pulses while the output capacitor is being charged. In other embodiments, the device may wait for one or more unsuccessful shocking attempts before delivering diaphragmatic pacing. At step 204, a shock pulse is delivered. At step 205, the ventricular sensing channel is checked to determine if the shock pulse was successful in terminating the ventricular fibrillation. If not, the device returns to step 202 to prepare for delivery of another shock pulse and possible diaphragmatic pacing. If the ventricular fibrillation was successfully terminated, the device checks for respiratory activity at step 206. If spontaneous breathing is occurring, the device returns to step 201. If respiratory arrest is still present, the device delivers a diaphragmatic pacing pulse during the ventricular refractory period after a ventricular sense at step 207 and then returns to step 205.

Although the invention has been described in conjunction with the foregoing specific embodiments, many alternatives, variations, and modifications will be apparent to those of ordinary skill in the art. Other such alternatives, variations, and modifications are intended to fall within the scope of the following appended claims.

1. An implantable cardiac rhythm management device, comprising:
a ventricular sensing channel for sensing ventricular depolarizations and generating a ventricular sense when a depolarization exceeds a specified threshold;
a ventricular shock channel for delivering a shock pulse;
a controller for detecting ventricular fibrillation from the rate of ventricular senses in the ventricular sensing channel;
a thoracic impedance channel for detecting respiratory activity;
a diaphragmatic pacing channel for delivering diaphragmatic pacing pulses; and,
wherein the controller is programmed to cause delivery of a shock pulse when ventricular fibrillation is detected and delivery of a diaphragmatic pacing pulse when no respiratory activity is detected.

2. The device of claim 1 wherein the diaphragmatic pacing channel is also used for delivering cardiac pacing pulses, a diaphragmatic pacing pulse being of higher energy than a cardiac pacing pulse.

3. The device of claim 2 wherein a diaphragmatic pacing pulse is on the order of 10 to 30 volts.

4. The device of claim 1 wherein the controller is programmed to begin charging an output capacitor of the ventricular shock channel when ventricular fibrillation is detected and to deliver a diaphragmatic pacing pulse while the output capacitor is charging if respiratory arrest is also detected.

5. The device of claim 1 wherein the controller is programmed to deliver a diaphragmatic pacing pulse when both respiratory arrest and ventricular fibrillation are detected only after one or more shock pulses are unsuccessful in terminating the ventricular fibrillation.

6. The device of claim 1 wherein the controller is programmed to deliver a diaphragmatic pacing pulse during a ventricular refractory period after a ventricular sense if respiratory arrest is detected while no ventricular fibrillation is present.

7. The device of claim 1 wherein the controller is programmed to:
begin charging an output capacitor of the ventricular shock channel when ventricular fibrillation is detected and to deliver a diaphragmatic pacing pulse while the output capacitor is charging if respiratory arrest is also detected; and,
deliver a diaphragmatic pacing pulse during a ventricular refractory period after a ventricular sense if respiratory arrest is detected after successful termination of ventricular fibrillation.

8. A cardiac rhythm management device, comprising:
means for monitoring ventricular activity in order to detect ventricular fibrillation;
means for monitoring respiratory activity;
means for delivering shock therapy upon detection of ventricular fibrillation; and,
means for delivering diaphragmatic pacing upon detection of respiratory arrest.

9. The device of claim 8 further comprising means for delivering diaphragmatic pacing during ventricular fibrillation while the shock therapy delivering means prepares to deliver a shock pulse.

10. The device of claim 8 further comprising means for delivering diaphragmatic pacing during a ventricular refractory period if ventricular fibrillation is not present.

11. A method for treating cardiac arrest by an implantable cardiac device, comprising:
monitoring a ventricular sensing channel in order to detect ventricular fibrillation;
monitoring a thoracic impedance channel in order to detect respiratory arrest;
delivering shock therapy through a ventricular shock channel upon detection of ventricular fibrillation; and,
delivering diaphragmatic pacing upon detection of respiratory arrest.

12. The method of claim 11 wherein diaphragmatic pacing is delivered as pacing pulses to the phrenic nerve.

13. The method of claim 12 where the pacing pulses are on the order of 10 to 30 volts.

14. The method of claim 12 further comprising beginning to charge an output capacitor of the ventricular shock channel when ventricular fibrillation is detected and to deliver a diaphragmatic pacing pulse while the output capacitor is charging if respiratory arrest is also detected.

15. The method of claim 11 further comprising deliver a diaphragmatic pacing when both respiratory arrest and ventricular fibrillation are detected only after one or more attempts with shock therapy are unsuccessful in terminating the ventricular fibrillation.

16. The method of claim 12 further comprising delivering a diaphragmatic pacing pulse during a ventricular refractory period after a ventricular sense if respiratory arrest is detected while no ventricular fibrillation is present.

17. The method of claim 11 further comprising:
beginning to charge an output capacitor of the ventricular shock channel when ventricular fibrillation is detected and to deliver a diaphragmatic pacing pulse while the output capacitor is charging if respiratory arrest is also detected; and,
delivering a diaphragmatic pacing pulse during a ventricular refractory period after a ventricular sense if respiratory arrest is detected after successful termination of ventricular fibrillation.
